(19) United States
(12) Patent Application Publication
(10) Pub. No.: US 2007/0082377 A1
(43) Pub. Date: Apr. 12, 2007

(54) Cytotoxicity assays

(76) Inventors: Michael R. Olin, Mounds View, MN (US); Gary L. Johnson, Bloomington, MN (US); Brian W. Lee, Minnetonka, MN (US)

Correspondence Address: VIKSNINS HARRIS & PADYS PLLP, P.O. BOX 111098, ST. PAUL, MN, 55111-1098, US

(21) Appl. No.: 11/246,007

(22) Filed: Oct. 7, 2005

(51) Int. Cl. G01N 1/30; G01N 33/48

(52) U.S. Cl. 435/40.5

(57) ABSTRACT

Certain embodiments of the invention provide methods and kits for determining cell viability and measuring cytotoxicity.

### BACKGROUND

Cell-mediated cytotoxicity is most often measured using the radioactive chromium (⁵¹Cr) release assay, first described by Brunner (Brunner, 1968). Assays of this type have been used to study a wide range of cell-mediated cytotoxicity driven processes, including: antibody dependent cell-mediated cytotoxicity of HIV-infected cells (Ward et al., 2004); cytotoxic T lymphocyte targeting of virus infected cells (Barber et al., 2003); cell-mediated lysis of tumor target cells (Brunner et al., 1968); cell targeting of melanoma cancer cells (Yang and Haluska, 2004); antibody +compliment-mediated lysis of nucleated cells (Klein and Perlmann; 1963); macrophage mediated cytotoxicity (Evans and Alexander, 1972); NK cytotoxicity (Rosenberg et al, 1974); graft versus host disease (Schwarer et al., 1994); T cell-mediated-insulin-dependent diabetes (Russell and Ley, 2002).

⁵¹Cr release assays are based upon the passive internalization and binding of ⁵¹Cr from sodium chromate by target cells. Lysis of the target cells by cell-mediated cytotoxicity results in the release of the radioactive probe into the cell culture supernatant (Brunner et al., 1968). While ⁵¹Cr release assays can provide useful quantitative information regarding the level of cell-mediated cytotoxicity present in the cell population(s), major concerns have been raised about the high cost of running the assays and the problems associated with exposure of laboratory workers to radioisotopes (Pross et al, 1986). Other issues associated with radioisotope use include the disposal of radioactive reagent and waste products and the detrimental effect of radioactivity emission on cell function (Jerome et al, 2003). The ⁵¹Cr release assay method is also quite labor intensive, susceptible to wide variations in radioactive labeling, and exhibits a relatively high level of spontaneous ⁵¹Cr release (Slezak, 1989). As a result of these draw-backs, other cytotoxicity assays are needed.

### SUMMARY OF CERTAIN EMBODIMENTS OF THE INVENTION

Certain embodiments of the present invention provide a method for determining the viability of a population of cells, including: combining with a sample that includes a first population of cells a first fluorescent probe that is a membrane stain, a second fluorescent probe that is a vital stain, and a third fluorescent probe that is a cell-permeable apoptosis-detection probe, and detecting the cells that bind to the first, second and/or third probe so as to determine the viability of the population of cells.

In some embodiments of the invention, the method may further include combining a second population of cells with the sample after the first population of cells has been combined with the membrane stain and the membrane stain binding reaction has been stopped. In some embodiments of the invention, the second population of cells includes immune cells. In some embodiments of the invention, the immune cells include lymphocytes and/or natural killer cells.

In some embodiments of the invention, the first population of cells is exposed to an experimental agent before the second or third probes are combined with the sample. In some embodiments of the invention, the experimental agent is an infectious agent, a pharmaceutical agent, or radiation.

In some embodiments of the invention, the method is performed in a single container. In some embodiments of the invention, the container is a test tube, a flask, a 96 well plate, or a tissue culture plate.

In some embodiments of the invention, the detection is performed using fluorescence spectroscopy, fluorescence microscopy, confocal fluorescence microscopy, fluorescence image analysis, flow cytometry, laser scanning cytometry, or a plate multi-well fluorescence reader. In some embodiments of the invention, the detection is via flow cytometry. In some embodiments of the invention, the detection step includes the detection of necrotic cells and apoptotic cells so as to determine the viability of the cells.

In some embodiments of the invention, the method may further include combining with the sample at least one additional probe that is a cell permeable granzyme B detection probe and/or a cell permeable granzyme A detection probe.

In some embodiments of the invention, the membrane stain is a thiol-reactive membrane stain or an amine-reactive membrane stain. In some embodiments of the invention, the membrane stain is carboxyfluorescein diacetate succinimidyl ester (CFSE).

In some embodiments of the invention, the vital stain is a cell-impermeant DNA stain. In some embodiments of the invention, the vital stain is 7-aminoactinomycin D (7-AAD).

In some embodiments of the invention, the cell-permeable apoptosis-detection probe is sulforhodaminyl-L-valylalanylaspartylfluoromethyl ketone (SR-VAD-FMK), or an ester or a salt thereof.

One embodiment of the invention provides a method for determining the viability of a population of cells, including: combining with a sample that includes a first population of cells carboxyfluorescein diacetate succinimidyl ester (CFSE); combining a second population of cells with the sample after the first population of cells has been combined with the CFSE and the CFSE binding reaction has been stopped; combining 7-aminoactinomycin D (7-AAD), and sulforhodaminyl-L-valylalanylaspartylfluoromethyl ketone (SR-VAD-FMK), or an ester or a salt thereof with the sample, and detecting via flow cytometry the cells that bind to the CFSE, 7-AAD, and/or SR-VAD-FMK so as to determine the viability of the population of cells.

In some embodiments of the invention, at least one population of cells is a preselected population of cells.

One embodiment of the invention provides a kit, including a first fluorescent probe that is a membrane stain, a second fluorescent probe that is a vital stain, and a third fluorescent probe that is a cell-permeable apoptosis-detection probe.

In some embodiments of the invention, the kit further includes instructions for using the kit to determine the viability of a population of cells via flow cytometry.

In some embodiments of the invention, the kit further includes a cell permeable granzyme B detection probe and/or a cell permeable granzyme A detection probe.

In some embodiments of the invention, the probes are packaged separately.

In some embodiments of the invention, the membrane stain is a thiol-reactive membrane stain or an amine-reactive membrane stain.

In some embodiments of the invention, the membrane stain is carboxyfluorescein diacetate succinimidyl ester (CFSE), the vital stain is 7-aminoactinomycin D (7-AAD), and the cell-permeable apoptosis-detection probe is sulforhodaminyl-L-valylalanylaspartylfluoromethyl ketone (SR-VAD-FMK), or an ester or a salt thereof.

In some embodiments of the invention, each kit may contain a single container, e.g., a vial, of each probe or multiple containers (e.g., 2, 3, 4, 5, 10, 15, or 20) of one or more of the probes. The amount of each probe per container can be any amount effective to label the target(s) effectively, e.g., about 50 μg, about 100 μg, about 130 μg, about 150 μg, about 200 μg, about 250 μg, or about 300 μg of each probe per container. In some embodiments, the probes may be packaged in individual containers in each kit. In some embodiments, at least two of the probes are packaged in the same container in the kit.

### BRIEF DESCRIPTION OF THE FIGURES

FIG. 1A depicts the forward scatter (FSC) versus side scatter (SSC) dot plot of unlabeled K562 cells and peripheral blood mononuclear cells (PBMCs). FIG. 1B depicts the CFSE staining (FL1; fluorescence 1) versus side scatter dot plot adjustment of the CFSE-labeled target cells to the third or fourth log of the FL1 scale. FIG. 1C is an example of forward scatter versus side scatter dot plot of unlabeled target cells and effector cells of the same size. FIG. 1D is an example of the CFSE (FL1) versus side scatter dot plot adjustment of the CFSE-labeled target cells to the third or fourth log of the FL1 scale when the target cells and effector cells are the same size.

FIG. 2A demonstrates the gating of live CFSE-stained target cells in a CFSE (FL1) versus 7-AAD (FL3; fluorescence 3) dot plot. Only CFSE-labeled target cells were used. FIG. 2B shows the adjustment of the photomultiplier tube (PMT) voltage so that the stained target cell population falls within the 3rd or 4th decade of the CFSE (FL1) versus 7-AAD (FL3) dot plot.

FIG. 3A is a CFSE (FL1) versus 7-AAD (FL3) dot plot using live, viable CFSE- and 7-AAD-stained target cells. FIG. 3B is a CFSE (FL1) versus 7-AAD (FL3) dot plot using heat killed CFSE- and 7-AAD-labeled target cells as a necrotic control.

FIG. 4A is a SR-VAD-FMK (FL2; fluorescence 2) versus 7-AAD (FL3) dot plot using viable target cells that have been labeled with CFSE and SR-VAD-FMK, adjusting the apoptosis negative population to account for background. FIG. 4B is a population of apoptosis-induced target cells that have been labeled with CFSE and SR-VAD-FMK on the SR-VAD-FMK (FL2) versus 7-AAD (FL3) dot plot.

FIG. 5 shows the final adjustment made on a CFSE (FL1) versus 7-AAD (FL3) dot plot by adding effector cells to the target cells that are labeled with CFSE, SR-VAD-FMK and 7-AAD. The instrument was set to collect events in the R2 region.

FIG. 6 shows the final analysis of the triple labeled (CFSE, SR-VAD-FMK and 7-AAD) target cells from the R2 region on a SR-VAD-FMK (FL2) versus 7-AAD (FL3) dot plot.

FIG. 7 depicts the effects of different ratios of effector cells to target cells (E/T ratios).

### DETAILED DESCRIPTION

Cytotoxic T lymphocytes (CTL) and natural killer (NK) cells are central mediators of the cell-mediated cytotoxicity process. These cells play an important role in protecting an organism against intracellular pathogens and cancerous tumor cells (Smyth, 2001; Lowdell, 1997). Autoimmunity and transplant rejection events are also driven by these activated effector cells (Russell, 2002). Upon recognition of foreign, cancerous, viral or bacterial infected cells by cytotoxic effector cells, apoptosis of the target cells is induced by one of two basic mechanisms (Henkart, 1994; Russell, 2002). One apoptosis induction pathway involves the ligation of Fas ligand to its receptor on the target cells, initiating an apoptotic caspase cascade. A second apoptosis induction pathway involves the utilization of cytoplasmic granules containing granzyme B and the pore forming protein, perforin (Trapani, 2003). Granzyme B and perforin alone have also been shown to induce apoptosis in target cells (Shi, 1992). Cell-mediated cytotoxicity is most often measured using the radioactive chromium (⁵¹Cr) release assay. However, the ⁵¹Cr release assays suffers from several drawbacks.

As a result of the drawbacks of the ⁵¹Cr release assay, other release assays that use non-radioactive compounds were developed. Some of these assays include assays which detects the loss of hydrolyzed (fluorescent) calcein-AM into the cell supernatant upon cytotoxic T lymphocyte attack, and endogenous lactate dehydrogenase (LDH) and alkaline phosphatase (AP) release assays (Lichtenfels et al., 1994, Korzeniewski et al., 1983, and Szekeres et al., 1981). However, these non-radioactive release assays suffer from high levels of spontaneous release of the tracker dye (calcein-AM) from the target cells or enzyme component (LDH, AP) from both target as well as effector cells that are used in the assay (Jerome et al., 2003).

Flow cytometric assays have been developed to overcome some of the negative aspects associated with the ⁵¹Cr release assay. These flow assays usually include a fluorescent membrane stain to label the target cells to differentiate them from the effector cell population, as well as a fluorescent vital stain to detect the dead and dying target cell population (Slezak and Horan, 1989, Radosevic et al., 1990, Hatam et al., 1994, Lee-MacAry et al., 2001, Hoppner et al., 2002, and Olin et al., 2005). Flow cytometric assays of this design show an excellent correlation with the ⁵¹Cr release assays that were run in parallel for comparison. Typical r² linear regression analysis values ran from 0.960 to 0.982 when % cytotoxicity values from the 2 assay formats were compared (Slezak and Horan, 1989, Radosevic et al., 1990, Lee-MacAry et al., 2001). Flow cytometric assays of this design have advantages over the traditional ⁵¹Cr release assays that include: elimination of hazardous radioactive components and waste products; no detrimental effects due to radiation on the effector population; shorter incubation times; lower cost; ability to differentiate subpopulations of cells (e.g., live targets, killed targets, live effectors, and dead effector cells); ability to monitor cytotoxicity effects on a single cell level; and to allow assessment of the viability of the effector cell population (Slezak and Horan, 1989, Radosevic et al., 1990, Hatam et al., 1994, and Kienzle et al., 2002).

Incorporation of an Annexin V-fluorescein isothiocyanate apoptosis detection probe (Annexin V-FITC) into the basic 2 fluorescent dye format allowed for the quantification of cells that exhibited negative propidium iodide (PI) vital staining properties (Fischer et al., 2002). Because of this feature, these cells would have been counted as part of the negative target cell population, even though they eventually would have registered as PI positive cells. This aspect would have lead to an underestimation of the amount of cytolytic activity occurring in the effector/target reaction system. Although the incorporation of the Annexin V-FITC apoptosis detection probe into the basic two-fluorescent dye format provides an addition to the information gathering capacity of the flow cytometric assay, certain features of the Annexin V-FITC binding with phosphatidyl serine (PS) can cause a false positive result. For example, inversion of the phosphatidyl serine molecules within the lipid bilayer of non-apoptotic cells as a normal cell membrane feature has been reported (Dillon et al., 2001). Based upon these Annexin V associated non-specific binding problems, the development of a different assay, for example, containing a different type of apoptosis detection probe was needed. Provided herein is the use of such a probe, e.g., the use of a cell permeable, fluorescence labeled inhibitor of caspase (FLICA) probe (see, e.g., U.S. Patent Application Pub. No. 2005/0136492).

Granzyme B-mediated cytotoxicity is the major mechanism by which CTL and NK cells eliminate intracellular pathogen infected cells, transformed cancer cells, as well as non-self cells in transplant rejection events. This apoptosis-inducing enzyme is the most abundant protease in the cytoplasmic granules (Poe et al., 1991). Combinatorial peptide library studies of granzyme B target specificity indicate that an optimal tetrapeptide recognition motif is IEPD (SEQ ID NO:1) and that an aspartic acid in the P1 position of the target sequence is critical for granzyme B activity (Thornberry et al., 1997).

Several substrate-based studies were performed using the chromogenic and fluorogenic reporter molecules paranitroanilide (pNA) and 7-amino-4-methylcoumarin (AMC) (Ewen et al., 2003, Rotonda et al., 2001, and Harris et al., 2000). However, in these studies, cell lysis was required in order to assess the granzyme B activity. Flow cytometric analysis of granzyme B levels in effector cell populations enables the investigator to identify the number of activated cytotoxic lymphocyte and/or natural killer effector cells capable of mounting a cytolytic response. Currently, no flow cytometric assay exists which would provide this type of granzyme B monitoring system. Certain embodiments of the present invention provide assays in which cell lysis is not required in order to assess the granzyme B activity.

As described herein, incorporation of a fluorophore-labeled, granzyme B specific, DAP inhibitor probe into a cytometric cytotoxicity assay allows for the detection of both necrotic and apoptotic target cell populations, as well as the identification of the percentage of effector cells involved in the cytolytic process. This flow cytometric assay configuration allows the investigator to monitor the dynamics of both the effector as well as target cell activities within the cell-mediated cytotoxic event. Fluorescent-labeled inhibitor probes containing a peptidylaminoalkanephosphonate (DAP) reactive group have been described (e.g., U.S. Pat. No. 5,916,877). The synthesis, mechanism, and use of serine-OH reactive DAP group inhibitors with granzymes and other proteases has been previously described (Oleksyszyn and Powers, 1994, and Kam et al., 2000).

Thus, certain embodiments of the present invention provide a highly sensitive flow cytometry-based method for the detection of cytotoxic T lymphocyte and natural killer cell activity in cell-mediated cytotoxicity assays.

Certain embodiments of the present invention provide fluorescent dye flow cytometric detection systems, e.g., a three or four color fluorescent dye flow cytometric detection system, useful for monitoring the outcome of cell-mediated cytotoxicity studies, e.g., in a single container, e.g., a test tube. The dyes and probe components can possess cell membrane permeability characteristics that allow the end user to accurately detect the membrane and apoptotic-associated changes of the cells resulting, e.g., from exposure to natural killer cell and cytotoxic T lymphocyte derived perforin and granzyme B cytolytic enzymes. The cell-mediated cytotoxicity detection system can be configured to contain: (1) a fluorescent dye capable of staining a cell population, e.g., a membrane stain; (2) a second fluorescent dye capable of detecting dead and membrane-compromised dying cells, i.e., a vital stain; and (3) a third fluorescent dye bound to a membrane permeant caspase inhibitor probe capable of detecting early apoptotic cells. The fluorescent dyes should typically emit at different wavelengths that can be differentiated by flow cytometric instruments.

In one configuration of the invention, the amine reactive, green fluorescing dye, carboxyfluorescein diacetate succinimidyl ester (CFSE) is used as the membrane stain. CFSE emits at 517 nm and binds with amine groups. In certain embodiments, membrane compromised dead and dying cells are identified using the red emitting vital stain, 7-aminoactinomycin D (7-AAD). This DNA binding dye emits at 647 nm, allowing for the use of a third fluorescent probe emitting in the orange wavelength region. Early apoptotic cells resulting, e.g., from effector cell perforin and granzyme B activity or the result of pharmaceutical reagents, can be detected by the use of fluorescence labeled inhibitors of caspases (FLICA) probes (see, e.g., U.S. Patent Application Pub. No. 2005/0136492). These fluorescent membrane permeant probes penetrate the cell membrane of live cells and covalently bind to active caspase enzymes in apoptotic cells (Bedner et al., 2000, Smolewski et al., 2001, and Smolewski et al., 2002). Sulforhodamine B, coupled to the fluoromethyl ketone (FMK) labeled tri-peptide caspase inhibitor sequence, VAD, yielded a poly caspase specific apoptosis detection probe (SR-VAD-FMK) emitting in the orange fluorescence region (586 nm) of the spectrum.

Use of a flow cytometric, three color fluorescence dye cell-mediated cytotoxicity detection assay system allows for the detection of four key cell populations: (1) live non-apoptotic cells (PI⁻/FLICA⁻); (2) live early apoptotic cells (PI⁻/FLICA⁺); (3) dead apoptotic cells (PI⁺/FLICA⁺); and (4) dead necrotic/late apoptotic cells (PI⁺/FLICA⁻). This system allows the investigator to include the previously over-looked live early apoptotic cell population with the mid-apoptotic-stage and late apoptotic/necrotic stage cytolytic activity values. Inclusion of these extra cytolysis cell counts provides a more accurate estimate of the true cell-mediated cytotoxic activity present in the mixed (effector+target) cell population.

A distinct advantage of this cell-mediated cytotoxicity assay invention over previously described necrosis/apoptosis cell-mediated detection systems relying on Annexin V binding for quantification of PI⁻ early apoptotic cells is the lack of false positives that is associated with the use of AnnexinV. Because of the incidence of Annexin V binding to activated, non-apoptotic lymphoid cells, its use as a marker of early apoptotic cell populations would result in the over estimation of the degree of cytolytic activity that was present in the effector/target cell cultures. As described herein, by using a cell permeant apoptosis detection probe, such as the active caspase specific FLICA probes, the present cytotoxicity detection system invention eliminates that serious assessment problem.

In certain embodiments, the invention can be used as a method for the analysis of mixed lymphocyte reaction (MLR), important for the detection of graph rejections. The recipient's lymphocytes, lymphocytes of interest or target cells, can be stained with the membrane stain (CFSE) allowing them to be analyzed separately from the donor cells. After incubation with donor cells, the cell mixture can be stained with a vital stain (e.g., 7-AAD) and an apoptosis detection probe (e.g., SR-VAD-FMK). This allows for a rapid, sensitive, analysis of compatibility between the two groups by flow cytometry.

Alternately, effector cells, or donor cells, may be stained with a membrane stain (e.g., CFSE) rather than staining the target cell population or recipient cell population. This separation allows for the analysis of cytolytic activity effects on the effector or donor cells when stained with the remaining reagents after incubation with the target or recipient cells.

Certain embodiments of the invention also provide a useful tool for reference laboratories for flow cytometric analysis of immune induction. By staining K562 cells, Yac cells, as well as other tumor cell lines used for the detection of natural killer activity, the cytotoxicity assay can be used as a sensitive method for the determination of immune activation.

The invention can also be used for research on the mechanisms involved in infectious diseases. Researchers and clinicians can use both in-vivo and in-vitro models to study immunological alterations as well as mechanisms following the introduction of infectious diseases.

The invention may also be used to assess the cytolytic effects of pharmaceutical reagents, therapeutics and/or radiation treatments on specific cell populations. For example, cells may be stained with a membrane stain (e.g., CFSE), then be incubated with the drug, receive radiation treatment or be incubated with other cells. Final analysis is made after adding the vital stain and apoptosis detection probe, and, optionally, the granzyme B detection probe. Under conditions where no identification or separation of cells is needed, the membrane stain can be omitted. Analysis is then made after treatment using only the vital stain and the apoptosis detection probe.

Another embodiment of the invention includes the use of a cell permeant probe for the detection of granzyme B. In this embodiment, the cell-mediated cytotoxicity detection system contains: (1) a first fluorescent dye capable of staining a cell population, i.e., a membrane stain; (2) a second fluorescent dye capable of being used to detect dead and membrane compromised dying cells, i.e., a vital stain; (3) a third fluorescent dye bound to a membrane permeant caspase inhibitor probe capable of being used to detect early apoptotic cells; and (4) a fourth fluorescent dye bound to a membrane permeant granzyme B inhibitor probe capable of being used to detect granzyme B. The fluorescent dyes should typically emit at different wavelengths that can be differentiated by flow cytometric instruments.

In this embodiment of the invention, the granzyme B probe can be used to identify the population of effector cells that are involved in the cell-mediated cytolytic activity. Incorporation of the fluorophore labeled granzyme B specific DAP inhibitor probe (IEPD-DAP) into the 3 color flow cytometric cytotoxicity assay format, thereby adding a fourth color, will enable detection of both necrotic and apoptotic target cell populations as well as allow for the calculation of the percentage of effector cells involved in the cytolytic process. A flow cytometric assay configured in this manner enables the investigator to monitor the dynamics of both the effector as well as target cell activities within the cell-mediated cytolytic event.

Effector cells may be stained with a membrane stain (e.g., CFSE). After incubation with the target cells, the granzyme B stain (e.g., IEPD-DAP) is added to determine the percentage of active natural killer cells in the effector cell population that are involved in the cytolytic process. This may also be used in conjunction with a vital stain (e.g., 7-AAD) and an apoptosis detection probe (e.g., SR-VAD-FMK) in a 4 color assay.

Alternately, the target cells may be stained as normal. The effector cells are then added and incubated. Following the incubation, the mixture is stained with an apoptosis probe (e.g., SR-VAD-FMK), a vital stain (e.g., 7-AAD) and a granzyme B stain (e.g., FAM-IEPD-DAP). Analysis may be run by gating on the target cells and analyzing the target cell population for necrotic, apoptotic and granzyme B positive cells. A second analysis may also be run by gating on the cells that were not stained with the membrane stain, providing the same information for the effector cell population.

Certain embodiments of the invention include the use of four fluorescent reagents. The four reagents typically emit at different wavelengths, allowing multiplexing. The first reagent is a membrane stain and is used to identify a population of cells. The second reagent is a vital stain and is used to identify necrotic or late-stage apoptotic cells that have a compromised cell membrane. The third reagent is a cell permeant probe used to detect early apoptosis. The fourth reagent is a cell permeant probe used to detect granzyme B.

The four reagents can be supplied individually or in combination for use in a cytotoxicity assay. There are several possible configurations that can be made with the reagents. Each should fluoresce at a different wavelength. Not all assay units contain all four reagent types.

In one embodiment of the invention, a population of cells (e.g., target cells or effector cells) in a solution is stained with a membrane stain. Following the staining of those cells, unstained cells (e.g., effector cells or target cells) are added to the solution, and the target and effector cells are incubated together. Following the incubation, a vital stain is added to the solution. As only the target or effector cells have been stained with the membrane stain, the populations of cells can be distinguished. A cell permeable apoptosis detection probe and/or a cell permeable granzyme B detection probe may also be added to the solution. Following incubation, the different population(s) of cells are detected and the viability of the cells can be determined, thereby determining the cytotoxicity. This method may be performed in a single container, such as a test tube.

In certain embodiments, these methods and assays can be used to assess the cytolytic effects of pharmaceutical agents, therapeutics, and/or radiation treatments on specific target cell populations.

Membrane Stains

The membrane stain is typically a detectable membrane stain, e.g., that can be used to detect a preselected population of cells. The membrane stain may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides).

The membrane stain is typically a stain that stains cell membranes, e.g., a cell permeant fluorescent dye with an active group that will form a covalent bond to proteins within the cell membrane, and as a result, be retained in the cell. The active groups are typically succinimidyl esters that bind with primary amines, or a methyl chloride that binds with free thiols, or a methyl bromide that binds with free thiols. Thus, certain membrane stains are thiol-reactive stains and certain membrane stains are amine-reactive stains. The dye may be fluorescent at all times or it may contain one or more acetate groups and become fluorescent when the membrane permeant probe enters the cell and esterase hydrolysis removes the acetate groups. In one embodiment, the membrane stain is used to stain the target and/or recipient cells (or to stain effector and/or donor cells), e.g., before the effector and target cells are added together (or donor and recipient cells) and incubated.

Certain embodiments of the invention provide methods and kits that contain at least one membrane stain to stain the cells, e.g., a preselected population of cells. In one embodiment of the invention, the amine reactive, green fluorescing dye, carboxyfluorescein diacetate succinimidyl ester (CFSE) is used as the membrane cell stain. CFSE has an optimal excitation at 475 nm and emits at 517 nm and binds with amine groups. The structure of CFSE is:

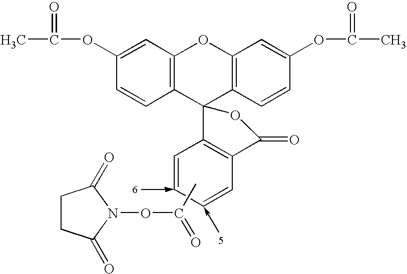

In another embodiment, the orange fluorescing dye, Cell Tracker Orange, is used as the membrane cell stain. This is a thiol reactive dye and will react with thiol groups in cell membrane and cytoplasmic proteins. Cell Tracker Orange has an optimal excitation at 541 nm and emits at 565 nm. The structure of Cell Tracker Orange is:

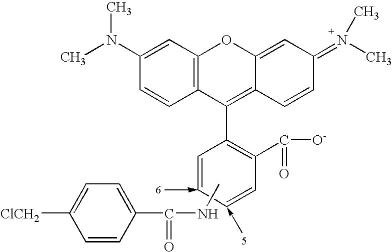

Other membrane stains include, but are not limited to, Cell Tracker Blue CMF₂HC, Cell Tracker Blue CMHC, Cell Tracker Blue CMAC, BODIPY 493/503 Methylbromide, SNARF-1 SE or BODIPY 630/650 Methylbromide (BODIPY Far Red). The structure of Cell Tracker Blue CMF₂HC is:

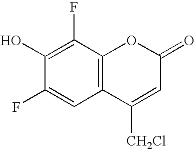

Table 1 provides a partial list of membrane stains, which can be purchased, for example, from Invitrogen, that can be used with certain embodiments of the invention.

TABLE 1
Examples of Membrane Stains

| Membrane Stain | Excitation | Emission | Reacts With |
|---|---|---|---|
| Cell Tracker Blue CMF₂HC | 371 nm | 464 nm | Thiol-reactive |
| Cell Tracker Blue CMAC | 353 nm | 466 nm | Thiol-reactive |
| Cell Tracker Blue CMHC | 372 nm | 470 nm | Thiol-reactive |
| BODIPY 493/503 | 493 nm | 503 nm | Thiol-reactive |
| Methylbromide | | | |
| Cell Tracker Green | 475 nm | 517 nm | Thiol-reactive |
| CFSE | 475 nm | 517 nm | Amine-reactive |
| Cell Tracker Green BODIPY | 522 nm | 529 nm | Thiol-reactive |
| Cell Tracker Orange | 541 nm | 565 nm | Thiol-reactive |
| SNARF-1 SE | 548 nm | 587 nm | Amine-reactive |
| BODIPY 630/650 | 630 nm | 650 nm | Thiol-reactive |
| Methylbromide | | | |

Vital Stains

The vital stain is typically a detectable vital stain, e.g., that can be used to detect a preselected population of cells. The vital stain may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides).

In certain embodiments of the invention, at least one stain is used as a vital stain to detect dead or necrotic cells, e.g., in a preselected population of cells. The vital stain can be a cell-impermeant DNA stain. This stain will enter membrane-compromised cells that have either died or are in very late stages of apoptosis, as the reagent will no longer be excluded from those cells. When this stain binds to or intercalates with DNA, it becomes detectable, e.g., fluorescent. In certain embodiments, the vital stain is added after the cells, e.g., effector and/or target cells or donor and/or recipient cells, are incubated, or pharmaceutical treatments or radiation treatments are made to the cells.

In one embodiment of the invention, 7-aminoactinomycin D (7-AAD) is used as the vital stain. 7-AAD is excited at 546 nm and emits at 647 nm when it is intercalated with the DNA. The structure of 7-AAD is:

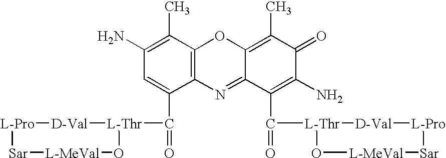

In another embodiment of the invention, one can use other vital stains, such as DNA binding stains, that are not cell permeant such as, but not limited to, TO-PRO-3, TO-PRO-5, SYTOX Blue, SYTOX Green or SYTOX Orange.

Table 2 provides a partial list of DNA stains, which can be purchased, for example, from Invitrogen, that may be used in this invention for the vital stain.

TABLE 2
Examples of Vital Stains

| Vital Stain | Excitation | Emission |
|---|---|---|
| SYTOX Blue | 431 nm | 480 nm |
| SYTOX Green | 504 nm | 523 nm |
| SYTOX Orange | 547 nm | 570 nm |
| 7-aminoactinomycin D (7-AAD) | 546 nm | 647 nm |
| TO-PRO-3 | 642 nm | 661 nm |
| TO-PRO-5 | 747 nm | 770 nm |

Apoptosis Detection Probes

The apoptosis detection probe is typically a detectable apoptosis detection probe, e.g., that can be used to detect a preselected population of cells. The apoptosis detection probe may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides).

Certain embodiments of the invention involve the use of at least one apoptosis detection probe that is cell permeant and capable of detecting cells in the early stages of apoptosis through later stages of apoptosis, e.g., in a preselected population of cells. In certain embodiments, a caspase affinity labeling probe is used as the apoptosis detection probe. This probe may be any agent capable of permeating the cell membrane and selectively binding, in a covalent manner, to one or more active caspases and facilitating their detection. In certain embodiments of the invention, the cell permeant apoptosis detection probe can be added after the effector and/or target cells, or donor and/or recipient cells are incubated, or pharmaceutical treatments or radiation treatments are made. Table 3 lists several caspase affinity labeling probes, which can be purchased, for example, from Invitrogen, and their corresponding caspase selectivity.

TABLE 3
Examples of Apoptosis Detection Probes

| Target Caspase | Product and Sequence |
|---|---|
| Poly-Caspase | Fluorescent Label-D-FMK |
| Poly-Caspase | Fluorescent Label-VD-FMK |
| Poly-Caspase | Fluorescent Label-VAD-FMK |
| Caspase-1 | Fluorescent Label-YVAD-FMK |
| Caspase-2 | Fluorescent Label-VDVAD-FMK |
| Caspase-3 and 7 | Fluorescent Label-DEVD-FMK |
| Caspases-4 and 5 | Fluorescent Label-WEHD-FMK |
| Caspase-6 | Fluorescent Label-VEID-FMK |
| Caspase-8 | Fluorescent Label-LETD-FMK, or IETD |
| Caspase-9 | Fluorescent Label-LEHD-FMK |
| Caspase-10 | Fluorescent Label-AEVD-FMK, or |
| | LELD |
| Caspase-13 | Fluorescent Label-LEED-FMK |

For example, such probes include fluorescent labels (e.g., fluorescein derivatives, sulforhodamine derivatives, Cy dye derivatives, BODIPY derivatives, coumarin derivatives, Quantum Dots, or any fluorescent dye that can be attached to an amino group directly or by linkers).

Other caspase affinity labeling probes may contain the same labels and a 1 to 5 amino acid sequence, but utilize an aldehyde modification of the aspartic terminal carboxyl group (HC═O), a chloromethyl ketone group (CH₂Cl), an acyloxy reactive group ((C═O)O—Ar, where Ar is [2,6-(CF₃)₂]benzoate and various derivative of same (Krantz et al., 1991, and Thornberry et al, 1994), or an aza-peptide epoxide modification of the aspartic acid (U.S. Patent Publication No. US 2004/0048327), or an aza-peptide Michael acceptor (Ekici et al., 2004).

For example, one caspase affinity labeling probe that can be used in certain embodiments of the present invention is a compound of formula I:
L₁-A₁-X₁—NH—CH(R₁′)C(═O)CH₂F (I)

wherein:

L₁ is a detectable group;

A₁ is a direct bond or a linker;

X₁ is absent, an amino acid, or a peptide; and

R₁′ is CH₂—COOH or CH₂CO₂R″, where R″ is methyl, ethyl, benzyl or t-butyl.

For a compound of formula (I), L₁ can in certain embodiments be a fluorescent label (e.g., sulforhodamine B, 5(6)-carboxyfluorescein, BODIPY dye, or Quantum Dot). For a compound of formula (I), X₁ may be a peptide having about 2 to 10 amino acids, e.g., about 2 to 4 amino acids (e.g., VA, YVA, DEV, LEE, LEH, VDVA (SEQ ID NO:2), or AEV). For a compound of formula (I), X₁ may be a natural amino acid (e.g., A, V, or E). For a compound of formula (I), R₁′ may be a methylene carboxy (ethanoic) side-chain (CH₂—COOH) as caspases typically have a requirement for aspartate in the P₁ position of the peptide substrate. In one embodiment, the carboxyl groups of all aspartic and glutamic amino acid residues exist as methyl esters of the carboxyl containing side-chains of —CH₂CO₂R, or CH₂CH₂CO₂R, where R is CH₃, other groups could include C₂H₅, C₄H₉, or CH₂C₆H₅ molecules.

A specific compound of formula (I) is sulforhodaminyl-L-valylalanylaspartylfluoromethyl ketone (SR-VAD-FMK), or 5(6)-carboxylfluoresceinyl-L-valylalanylaspartylfluoromethyl ketone (FAM-VAD-FMK); or an ester thereof, or a salt thereof. The structure of sulforhodaminyl-L-valylalanylaspartylfluoromethyl ketone (SR-VAD-FMK) is the following:

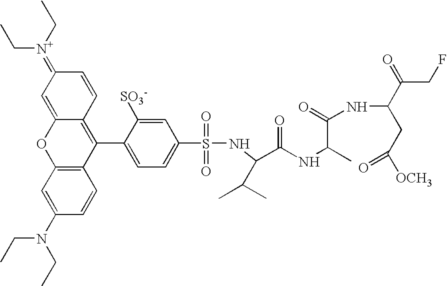

There are two main classes of a-amino acids: “natural” and “unnatural” α-amino acids. Additionally there are a wide variety of β-amino acids, homologues of amino acids and molecules that mimic amino acids, such as isosteres.

“Natural amino acids” refers to the naturally occurring α-amino acid molecules typically found in proteins. These include glycine, alanine, valine, leucine, isoleucine, serine, methionine, threonine, phenylalanine, tyrosine, tryptophan, cysteine, proline, histidine, aspartic acid, asparagine, glutamic acid, glutamine, arginine, and lysine. “Natural amino acids” also exist in nature that are not typically incorporated into naturally occurring proteins. Examples of these amino acids are: ornithine, γ-carboxyglutanic acid, hydroxylysine, citrulline, kynurenine, 5-hydroxytryptophan, norleucine, norvaline, hydroxyproline, phenylglycine, sarcosine, γ-aminobutyric acid and many others.

“Unnatural amino acids” are those amino acids that are not found in nature and may be obtained by synthetic means well known to those schooled in amino acid and peptide synthesis. Examples of this class, which numbers in the many thousands of known molecules include: (t-butyl)glycine, hexafluoro-valine, hexafluoroleucine, trifluoroalanine, β-thienylalanine isomers, β-pyridylalanine isomers, ring substituted aromatic amino acids, at the ortho, meta, or para position of the phenyl moiety with one or more of standard groups of organic chemistry such as: fluoro-, chloro-, bromo-, iodo-, hydroxy-, methoxy-, amino-, nitro-, alkyl-, alkenyl-, alkynyl-, thio-, aryl-, heteroaryl- and the like.

It will be appreciated that amino acids and peptides can exist in L- or D-forms (enantiomers) and that certain amino acids with more than one chiral center, such as threonine, may exist in diastereomeric form. Further, when linked together in peptide chains, a mixture of L- and D- amino acids may be chosen to confer desired properties known in the art. Therefore, enantiomers, diastereomers and mixtures of these types are included in the scope of certain embodiments of the invention.

Further, unnatural amino acids may exhibit other types of isomerism, such as positional and geometrical isomerism. These types of isomerism, coupled with or independent of optical isomerism, are also included in these claims.

In one embodiment, the term “amino acid,” includes the residues of the natural amino acids (e.g., Ala (A), Arg (R), Asn (N), Asp (D), Cys (C), Glu (E), Gln (Q), Gly (G), His (H), Hyl, Hyp, Ile (I), Leu (L), Lys (K), Met (M), Phe (F), Pro (P), Ser (S), Thr (T), Trp (W), Tyr (Y), and Val (V)) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, omithine, citruline, -methyl-alanine, para-benzoylphenylalanine, phenylglycine, propargylglycine, sarcosine, and tert-butylglycine). When X is an amino acid in a compound of formula I, the amino terminus is on the left and the carboxy terminus is on the right.

The term “peptide” refers to a sequence of 2 to 20 amino acids and/or peptidyl residues. In one embodiment, a peptide includes 2 to 10, e.g., 2 to 5, e.g., 2 to 4, amino acids. When X is a peptide in a compound of formula I, the amino terminus is on the left and the carboxy terminus is on the right.

The term “detectable group”, as it relates to compounds of formula I, includes any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, or plate multi-well fluorescence reader. Thus, suitable groups include florescent labels (e.g., fluorescein, rhodamines, Cy dyes, Bodipys, sulforhodamine 101, Quantum Dots, phycobiliproteins, etc.).

The nature of the “linker” is not critical provided the final compound of formula I has suitable properties (e.g., suitable solubility, cell toxicity, cell permeability, and ability to selectively react with the targeted caspase protease group) for its intended application. The linker, denoted by A₁, in the case where A₁ can simply be a covalent bond, the detectable group (L₁) is attached directly to the N-terminal amino group of the peptide or amino acid (e.g., amide linkage L-(C═O)—NH—R). A₁ can also be any member of the class of linkers well known to the art. Linkers are typically about 4-18 atoms long, including carbon, nitrogen, oxygen or sulfur atoms. Specific examples of linkers include ε-aminocaproic acid (6 atoms), di-ε-aminocaproic acid (12 atoms), oligomers of ethylene glycol (—O—(CH₂CH₂O)ₙCH₂CH₂—, where n=0-5); or di- and triamines separated by 2 to 6 methylene groups, for example: —HN(CH₂)ₙ—NH(CH₂)ₘ—NH(CH₂)₀— where n, m and o are integers from 0 to 6. Typical linkers include ester (—OC(═O)—), thioester (SC(═O)—), thionoester (—OC(═S)—), carbonyl (—C(═O)—), and amide (—NHC(═O)—) groups, as well as divalent phenyl groups, and a 1 to 10 membered carbon chain, which chain can optionally include one or more double or triple bonds, and which chain can also optionally include one or more oxy (—O) or thioxy (—S—) groups between carbon atoms of the chain. A preferred linker is a simple amide linkage ((—NHC(═O)—) or —C(═O)NH—) facilitated by an activated carboxyl-N-hydroxysuccinimide leaving group coupling system.

Granzyme B Detection Probes

The granzyme B detection probe is typically a detectable granzyme B detection probe, e.g., that can be used to detect a preselected population of cells. The granzyme B detection probe may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides).

The granzyme B detection probe may, e.g., be cell permeant and capable of detecting granzyme B activity, e.g., that can be used to detect granzyme B activity in a preselected population of cells. In certain embodiments, a granzyme B specific serine protease affinity labeling probe, including any agent capable of permeating the cell membrane and selectively binding, in a covalent manner, to active granzyme B and facilitating its detection, is used. The granzyme B probe may in certain embodiments be added after the effector and/or target cells, or donor and/or recipient cells are incubated, or pharmaceutical treatments or radiation treatments are made.

In certain embodiments, a flow cytometry compatible granzyme B assay using a membrane permeant, granzyme B probe, allows for the detection and quantification of granzyme B activity in whole living cells. A probe of this type includes in certain embodiments a tetrapeptide granzyme B target amino acid sequence flanked on one side by a serine hydroxyl-reactive functional group and a fluorophore on the other. Fluorescent labeled inhibitor probes containing a peptidylaminoalkanephosphonate (DAP) reactive group have been described (e.g., U.S. Pat. Nos. 5,543,396; 5,681,821; and 5,916,877). The synthesis, mechanism, and use of serine-OH reactive DAP group inhibitors with granzymes and other proteases has been previously described (Oleksyszyn and Powers, 1994, Kam et al., 2000). The structure of an example of a granzyme B probe is 5(6)-carboxylfluoresceinyl-hexaminocaproyl-L-leucyl-L-glutamyl -L-prolyl-L-aspartylpeptidylaminoalkanephosphonate (FAM-IEPD-DAP):

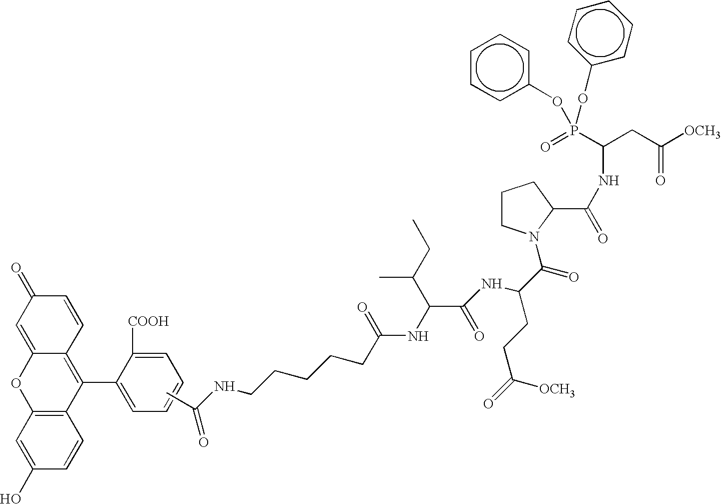

Incorporation of a fluorophore labeled, granzyme B specific, DAP inhibitor probe into the 3 color flow cytometric cytotoxicity assay format described herein enables detection of both necrotic and apoptotic target cell populations as well as identifies the percentage of effector cells involved in the cytolytic process. A flow cytometric assay configuration of this type enables the investigator to monitor the dynamics of both the effector as well as target cell activities within the cell-mediated cytolytic event.

Table 4 provides a list of different possible fluorescent labels, which can be purchased, for example, from Invitrogen, for the apoptosis and granzyme B detection probes.

TABLE 4
Examples of Fluorescent Labels

| Fluorescent Label for Apoptosis | | |
|---|---|---|
| or Granzyme B Probes | Excitation | Emission |
| Alexafluor 350 (AF 350) | 346 nm | 442 nm |
| Carboxyfluorescein (FAM) | 488 nm | 517 nm |
| BODIPY R6G | 528 nm | 550 nm |
| Sulforhodamine B (SR) | 568 nm | 584 nm |
| BODIPY TR | 589 nm | 617 nm |
| Cy5 | 649 nm | 670 nm |
| Cy7 | 743 nm | 767 nm |

There are several possible combinations of the various reagents to provide 3 and 4 color configurations of the invention. Table 5 lists some combinations, but not all combinations, for a 3 color configuration. The granzyme B probe may be added to these combinations.

TABLE 5
Examples of Combinations of Probes

| Membrane Stain | Vital Stain | Apoptosis Probe |
|---|---|---|
| CFSE (475/517) | 7-AAD (546/647) | SR-VAD-FMK (568/584) |
| Cell Tracker Orange | 7-AAD (546/647) | FAM-VAD-FMK |
| (541/565) | | (488/517) |
| Cell Tracker Orange | TO-PRO-3 (642/661) | FAM-VAD-FMK |
| (541/565) | | (488/517) |
| Cell Tracker Blue | SYTOX Green | Cy5-VAD-FMK |
| (371/464) | (504/523) | (649/670) |

Table 6 lists some combinations, but not all combinations, for a 4 color configuration.

TABLE 6
Examples of Combinations of Probes

| | | | Granzyme B |
|---|---|---|---|
| Membrane Stain | Vital Stain | Apoptosis Probe | Probe |
| CFSE (475/517) | 7-AAD | SR-VAD-FMK | AF350-IEPD- |
| | (546/647) | (568/584) | DAP (346/442) |
| Cell Tracker | 7-AAD | FAM-VAD-FMK | Cy7-IEPD-DAP |
| Orange (541/565) | (546/647) | (488/517) | (743/767) |
| Cell Tracker Blue | 7-AAD | SR-VAD-FMK | FAM-IEPD-DAP |
| (371/464) | (546/647) | (568/584) | (488/517) |
| Cell Tracker Blue | SYTOX Green | Cy5-VAD-FMK | SR-IEPD-DAP |
| (371/464) | (504/523) | (649/670) | (568/584) |

Granzyme A Probes

Certain embodiments of the present invention also provide for the use of probes for detecting granzyme A activity, e.g., that can be used to detect granzyme A activity in a preselected population of cells. The granzyme A detection probe is typically a detectable granzyme A detection probe, e.g., that can be used to detect a preselected population of cells. The granzyme A detection probe may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides). These probes include, for example: fluorescent label-PFR-DAP (Joseph A. Trapani (2001) Granzymes: A family of lymphocyte granule serine proteases, Genome Biology, 2, 3014.1-3014.7); fluorescent label-GPR-DAP (ibid); and fluorescent label-R-DAP (Clara Hink-Schauer et al., (2003) Crystal structure of the apoptosis-inducing human granzyme A dimmer, Nature Structural Biology, 10, 535-540).

The invention will now be illustrated by the following non-limiting Examples.

### EXAMPLE 1

### Protocol for Using 3 Color Cytotoxicity Assay Kit

This kit used in this example contained lyophilized vials of CFSE, 7-AAD and SR-VAD-FMK and a bottle of 10× assay buffer. The reagents were prepared for use as follows.

Carboxyfluorescein diacetate succinimidyl ester (CFSE) Each vial of lyophilized CFSE (50 μg) was reconstituted with 200 μL DMSO. This yielded a 2500× stock concentrate. If used immediately, the 2500× CFSE stock was diluted 1:250 in sterile PBS, pH 7.4. For example, 10 μL of the 2500× CFSE stock was added to 2.490 mL sterile PBS. This yielded 2.500 mL of the 10× CFSE working solution. Any unused 2500× CFSE stock was stored at ≦−20° C. (Step 3). Typically, the 10× CFSE working solution should be used as soon as it is prepared.

7-aminoactinomycin D (7-AAD)

Each vial of lyophilized 7-AAD (260 μg) was reconstituted with 260 μL DMSO. This yielded a 210× stock concentrate. The 210× 7-AAD stock was diluted 1:10 in sterile PBS, pH 7.4 to form the 21× 7-AAD working solution. For example, 40 μL of the 210× 7-AAD stock was added to 360 μL sterile PBS, which yielded 400 μL of the 21× 7-AAD working solution. Any unused 210× 7-AAD stock was stored at ≦−20° C. Typically, the 21× 7-AAD working solution should be used as soon as it is prepared.

Sulforhodaminyl-L-valylalanylaspartylfluoromethyl ketone (SR-VAD-FMK)

Each vial of lyophilized SR-VAD-FMK (131 μg) was reconstituted with 100 μL DMSO, which yielded a 252× stock concentrate. If all the 252× SR-VAD-FMK stock was immediately used, a 40× working solution was prepared by adding 530 μL sterile PBS, pH 7.4 or RPMI to each vial to form 630 μL of the 40× SR-VAD-FMK working solution. If all of the 252× SR-VAD-FMK stock was not used, it was diluted 1:6.3 by adding 10 μL of the 252× SR-VAD-FMK stock to 53 μL sterile PBS, pH 7.4 or RPMI. Any unused 252× SR-VAD-FMK stock was stored at ≦−20° C. Typically, the 40× SR-VAD-FMK working solution should be used as soon as it is prepared.

10× assay buffer

A 10× assay buffer was prepared according to the following recipe:

NaCl: 87.66 g/L (1.5 M);

Na2HPO4•12 H2O: 60.53 g/L;

NaH2PO4•2H2O: 4.84 g/L (0.2 M phosphate);

pH is 6.9;

1× solution (when diluted in DI H2O) has a final pH of 7.4.

If necessary, the 10× concentrate was gently warmed, without allowing it to boil, to completely dissolve any salt crystals that may have come out of solution. The 10× assay buffer was diluted 1:10 in sterile/endotoxin-free DI H₂O. For example, 10 mL 10× assay buffer was added to 90 mL sterile/endotoxin-free DI H₂O to make 100 mL. The solution was stirred for 5 minutes or until all crystals have dissolved. 1× assay buffer may be stored, e.g., for up to 5 days at 2-8° C.

Target Cell Staining (K562 Cells) Protocol

The number of target cells was adjusted to 1-2×10⁶ cells in 1.0 mL of 1× assay buffer by either dilution or concentration. The cells were concentrated by gentle centrifugation at <300× g for 5 minutes to pellet the cells. The cells were then resuspended in 1.0 mL 1× assay buffer. The target cells were washed 2 times with 1× assay buffer and resuspended into 1.8 mL of 1× assay buffer after the final wash. The cells were stained by adding 200 μL of the 10× CFSE working solution to the 1.8 mL suspension of target cells. The stained target cells were gently vortexed and allowed to incubate for 15 minutes at room temperature. Immediately, 1 mL of cell culture media was added to stop the CFSE binding reaction (any unbound CFSE will bind to the protein in the cell culture media). The CFSE-labeled target cells were centrifuged at <300×g for 5 minutes to pellet the cells. The CFSE-labeled target cells were resuspended in 2-3 ml of cell culture media and incubated at 37° C. for 30 minutes in a CO₂ incubator.

Effector Cell and Target Cell Incubation

IK562 target cells were labeled with CFSE and adjusted to 1.5×10⁴ cells per tube. Effector cells were added to fmal effector:target cell ratios of 0:1, 12.5:1, 25:1, 50:1, and 100:1. The effector cells were added to the target cells, creating a final sample size of 400 μL (forming the effector cell+target cell mixture). If the combined effector cell+target cell sample volume was larger than 400 μL, it was gently centrifuged at <300×g, 5 minutes to remove excess media. Cells were then incubated for 4 hours at 37° C. to allow the cytolytic activity to progress.

Detection of Apoptotic Cells

Apoptotic cells were detected via caspase activity using SR-VAD-FMK. If an effector cell+target cell sample volume of 400 μL was used, 10 μL of the 40× SR-VAD-FMK working solution as prepared above was added. If a different volume was used, the 40× working solution was added at a 1:40 v/v ratio. The SR-VAD-FMK probe was carefully mixed in the cell suspension tube and incubated for 45 minutes at 37° C. protected from light.

Detection of Dead or Necrotic Cells

Dead or necrotic cells were detected using the vital stain 7-AAD. This dye penetrates the structurally-compromised cell membranes of the dead and dying cells and complexes with DNA. Immediately following the 45 minute incubation for apoptosis detection, 20 μL of the 21× 7-AAD working solution was added to the 410 μL mixture of effector cells, target cells and SR-VAD-FMK probe. The tube was mixed or gently vortexed, placed in an ice bath and incubated for 15 minutes protected from light just prior to performing the flow analysis.

Preparation of Controls Used to Set Up the Flow Cytometer

To set the flow cytometer to most accurately analyze the results involves certain controls: (1) non-stained effector cells and non-stained target cells; (2) non-stained target cells; (3) viable CFSE-stained target cells; (4) viable dual stained CFSE and SR-VAD-FMK stained target cells; (5) apoptosis-induced dual stained CFSE and SR-VAD-FMK stained target cells; (6) viable dual stained CFSE and 7-AAD target cells; and (7) killed dual stained CFSE and 7-AAD target cells, prepared, for example, by immersing the tube of cells for 3 to 6 minutes in a 56° C. water bath, cooling to room temperature, and adding 7-AAD. Using these controls, the proper compensation was set.

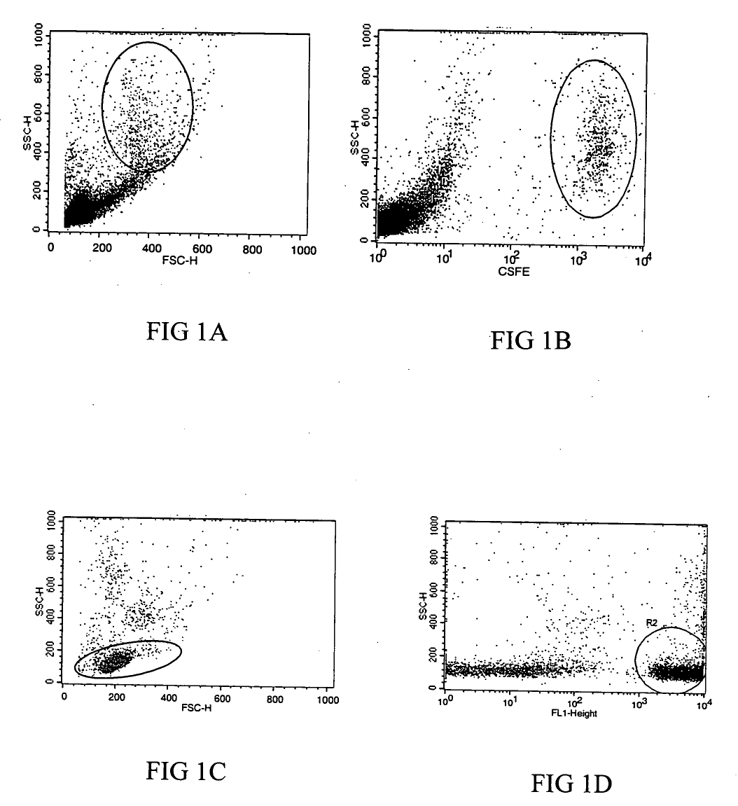

To ensure proper gating on the target cell population, using the flow software, a forward scatter versus side scatter dot plot (FIG. 1A) or CFSE (FL1) versus side scatter dot plot (FIG. 1b) was prepared prior to analyzing CFSE target cells. This allows for the analysis of only the target cell population. A CFSE (FL 1) versus side scatter dot plot becomes important when target cells are the same size as effector cells (FIG. 1C). When Mycobacterium-infected monocytes were used as target cells, the target cells were easily distinguished from effector lymphocytes by creating a CFSE (FL 1) versus side scatter dot plot (FIG. 1D). The data was saved to ensure that target cells were properly gated during analysis.

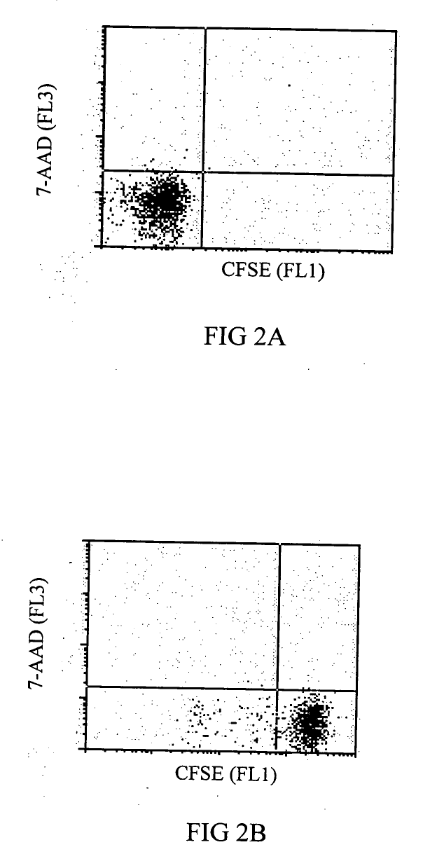

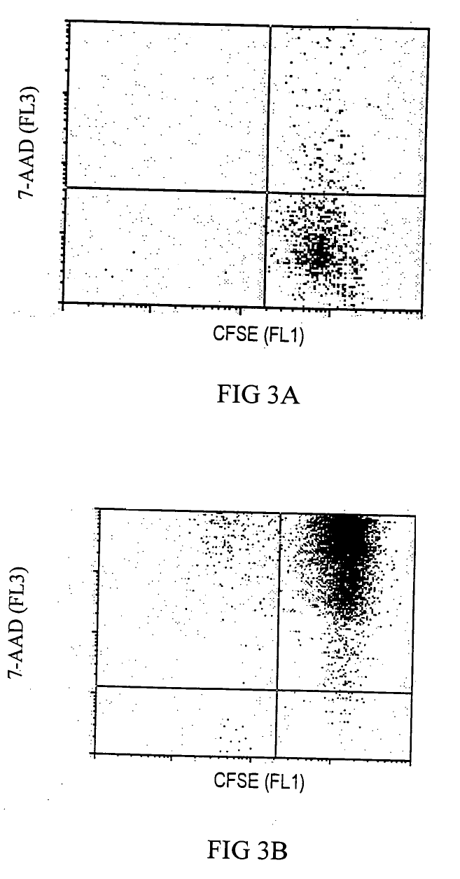

A CFSE (FL1) versus 7-AAD (FL3) dot plot was designed to analyze the gated population, as demonstrated in FIG. 1 (target population) to analyze the CFSE stained target cells and gate around the target cell population (FIG. 2A) and ensuring proper CFSE staining. The PMT voltage was adjusted so that the stained target cell population falls within the 3^{rd} or 4^{th} decade (FIG. 2B). The CFSE (FL1) versus 7-AAD (FL3) plot was used as designed above to analyze live (viable) CFSE stained target cells that were also been stained with 7-AAD (FIG. 3). This allowed for the compensation of viable and non-viable cells. Heat killed target cells were used as a necrotic control (FIG. 3B).

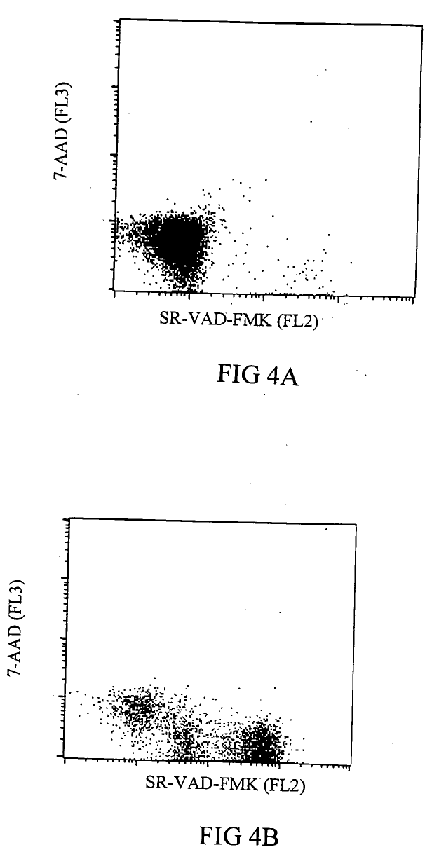

To ensure proper compensation for SR-VAD-FMK (FL2), a dot plot of SR-VAD-FMK (FL2) versus 7-AAD (FL3) was designed. Live CFSE-stained target cells that have also been stained with SR-VAD-FMK were used (FIG. 4). Healthy target cells that were stained with SR-VAD-FMK were used as negative controls to compensate the instrument (FIG. 4A). This adjusted the negative population to account for background. Apoptosis was induced to use as positive (apoptotic) control cells that have been stained with SR-VAD-FMK to produce FIG. 4B. To compensate the instrument to distinguish necrosis from apoptosis, CFSE stained target cells that have also been stained with SR-VAD-FMK and 7-AAD were analyzed. A SR-VAD-FMK (FL2) versus 7-AAD (FL3) dot plot was designed.

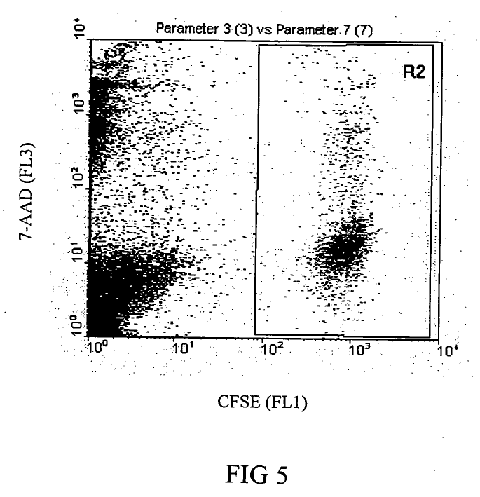

As a final adjustment, effector cells were added to CFSE-stained target cells. Using a CFSE (FL1) versus 7-AAD (FL3) dot plot, a region around the CFSE stained target cells (R2) was drawn, as shown in FIG. 5. Only events in this R2 region were collected for data analysis.

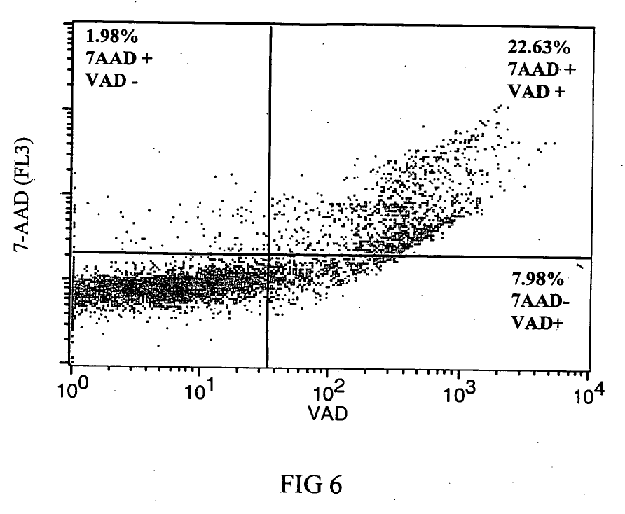

Data Analysis. Following final adjustments of the instrument, data was analyzed using the plots as described above. SR-VAD-FMK (FL2) versus 7-AAD (FL3) determined total cytotoxicity (FIG. 6).

EXAMPLE 2

The protocol in EXAMPLE 1 was followed using pig PBMCs as the effector cells and K562 cells as the target cells. The purpose of this experiment was to determine the effects of different ratios of effector cells to target cells and demonstrate an increase in total cytotoxicity when the apoptotic cell population was added to the necrotic cell population.

The K652 target cells were labeled with CFSE as described in EXAMPLE 1. After labeling, the cells were adjusted to a concentration of 1.0×10⁵ cells/mL and of this, 2.0×10⁴ cells (200 μL) were placed into sterile FACS tubes. The effector cells were counted and added to the FACS tubes providing ratios of 12.5:1, 25:1, 50:1 and 100:1 of effector cells to target cells. A control with no effector cells was also run. The volumes in all FACS tubes were adjusted to 400 μL with cell culture media. The cells were then incubated for 4 hours in a in a 37° C., 5% CO₂ incubator.

Forty-five minutes prior to the end of the incubation time, 10 μL of the 40× SR-VAD-FMK working solution, as prepared in EXAMPLE 1, was added to each of the FACS tubes containing 400 μL of effector cells and target cells to detect apoptosis. The FACS tubes were placed into the 37° C., 5% CO₂ incubator for the final 45 minutes of incubation time.

Upon completion of the 4 hour incubation in the 37° C., 5% CO₂ incubator, necrotic cells were stained by adding 20 μL of the 21× 7-AAD working solution, as prepared in EXAMPLE 1, to the 410 μL mixture of effector cells, target cells and SR-VAD-FMK probe. The FACS tubes were then incubated on ice for 15 minutes and read on a flow cytometer set up according to the protocol in EXAMPLE 1.

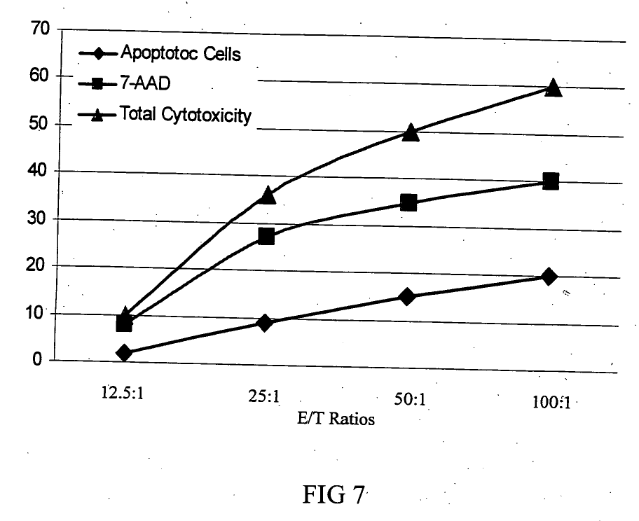

The results (Table 7) demonstrate an increase in cytotoxic effects with the increase in effector cell to target cell ratios, and that cytotoxicity assays that measure only necrotic cells miss a significant amount of the total cytotoxicity when compared with this assay that also detects early apoptosis. The results are depicted graphically in FIG. 7.

TABLE 7

| | Effector:Target Ratio | | | |
|---|---|---|---|---|
| | 12.5:1 | 25:1 | 50:1 | 100:1 |
| % Apoptotic cells | 2% | 9% | 15% | 20% |
| % Necrotic cells | 8% | 28% | 35% | 40% |
| % Total cytotoxicity | 10% | 37% | 50% | 60% |
| % Increase in cytotoxicity when | 25% | 33% | 43% | 50% |
| detecting apoptosis | | | | |

### Cited Documents

Barber, D. L., Wherry, E. J., and Ahmed, R. (2003) Cutting Edge: Rapid in vivo killing by memory CD8 T cells. J. Immunol. 171, 27-31.

Bedner, E., Smolewski, P., Amstad, P., and Darzynkiewicz, Z. (2000) Activation of caspases measured in situ by binding of fluorochrome-labeled inhibitors of caspases (FLICA): correlation with DNA fragmentation. Exp. Cell Res. 259, 308-313.

Brunner, K. T., Mauel, J., Cerottini, J. C., and Chapuis, B. (1968) Quantitative assay of the lytic action of immune lymphoid cells on 51-Cr-labeled allogenic target cells in vitro; inhibition by isoantibody and by drugs. Immunology 14, 181-196.

Dillon, S. R., Constantinescu, A., and Schlissel, M. S. (2001) Annexin V binds to positively selected B cells. J. Immunol. 166, 58-71.

Ekici et al., J. Med Chem, 2004 47:1889-1892.

Evans, R., and Alexander, P. (1972) Mechanism of immunologically specific killing of tumour cells by macrophages. Nature 236, 168.

Ewen, C., Kane, K. P., Shostak, I., Griebel, P. J., Bertram, E. M., Watts, T. H., Bleackly, R. C., and McElhaney, J. E. (2003) A novel cytotoxicity assay to evaluate antigen-specific CTL responses using a colorimetric substrate for granzyme B. J. Immunol. Methods 276, 89-101.

Fischer, K., Andreesen, R., and Mackensen, A. (2002) An improved flow cytometric assay for the determination of cytotoxic T lymphocyte activity. J. Immunol. Methods 259, 159-169.

Harris, J. L., Backes, B. J., Leonetti, F., Mahrus, S., Ellman, J. A., and Craik, C. S. (2000) Rapid and general profiling of protease specificity by using combinatorial fluorogenic substrates libraries. PNAS 97, 7754-7759.

Hatam, L., Schuval, S., and Bonagura, V. R. (1994) Flow cytometric analysis of natural killer cell function as a clinical assay. Cytometry 16, 59-68.

Henkart, P. A., (1994) Lymphocyte-mediated cytotoxicity: two pathways and multiple effector molecules. Immunity 1, 343-346.

Hink-Schauer et al., (2003) Crystal structure of the apoptosis-inducing human granzyme A dimmer, Nature Structural Biology, 10, 535-540.

Hoppner, M., Luhm, J., Schlenke, P., Koritke, P., and Frohn, C. (2002) A flow-cytometry based cytotoxicity assay using stained effector cells in combination with native target cells. J. Immunol. Methods 267, 157-163.

Jerome, K. R., Sloan, D. D., and Aubert, M. (2003) Measurement of CTL-induced cytotoxicity: The caspase 3 assay. Apoptosis 8, 563-571.

Kam, C., Hudig, D., and Powers, J. C. (2000) Granzymes (lymphocyte serine proteases): characterization with natural and synthetic substrates and inhibitors. Biochimica et Biophysica Acta 1477, 307-323.

Kienzle, N., Olver, S., Buttigieg, K., and Kelso, A. (2002) The fluorolysis assay, a highly sensitive method for measuring the cytolytic activity of T cells at very low numbers. J. Immunol. Methods 267, 98-108.

Klein, G., and Perlmann, P. (1963) In vitro cytotoxic effect of isoantibody measured as isotope release from labeled target cell DNA. Nature 199, 451.

Korzeniewski, C., and Callewaert, D. M. (1983) An enzyme-release assay for natural cytotoxicity. J. Immunol. Methods 64, 313-320.

Krantz et al., Biochemistry, 1991, 30:4678-4687

Lee-MacAry, A. E., Ross, E. L., Davies, D., Laylor, R., Honeychurch, J., Glennie, M. J., Snary, D., and Wilkinson, R. W. (2001) Development of a novel flow cytometric cell-mediated cytotoxicity assay using the fluorophores PKH-26 and TO-PRO-3 iodide. J. Immunol. Methods 252, 83-92.

Lichtenfels, R., Biddison, W. E., Schulz, H., Vogt, A. B., and Martin, R.(1994) CARE-LASS (calcien-release-assay) an improved fluorescence-based test system to measure cytotoxic T lymphocyte activity. J. Immunol. Methods 172, 227-239.

Lowdell, M. W., Ray, N., Craston, R., Corbett, T., Deane, M., and Prentice, H. G. (1997) The in vitro detection of anti-leukaemia-specific cytotoxicity after autologous bone marrow transplantation for acute leukaemia. Bone Marrow Transplant. 19, 891-897.

Oleksyszyn, J., and Powers, J. C., (1994) Amino acid and peptide derivatives as specific inhibitors of serine peptidases. Methods Enzymol. 244, 423-441.

Olin, M. R., Choi, K. H., Lee, J., and Molitor, T. W. (2005) γδ T-lymphocyte cytotoxic activity against Mycobacterium bovis analyzed by flow Cytometry. J. Immunol. Methods 297, 1-11.

Poe, M., Blake, J. T., Boulton, D. A., Gammon, M., Sigal, N. H., Wu, J. K., Zweerink, H. J. (1991) Human cytotoxic lymphocyte granzyme B. Its purification from granules and the characterization of substrate and inhibitor specificity. J. Biol. Chem. 266, 98-103.

Pross, H., Callewaert, D., and Rubin, P. (1986) Assays for NK cell cytotoxicity-their values and pitfalls. In: E. Lotzova and R. B. Herberman (Eds.) Immunobiology of Natural Killer Cells, Vol. I. CRC Press, Boca Raton, Fla., ch. 1, p.1.

Radosevic, K., Garritsen, H. S. P., Van Graft, M., De Grooth, B. G., and Greve, J. (1990) A simple and sensitive flow cytometric assay for the determination of the cytotoxic activity of human natural killer cells. J. Immunol. Methods 135, 81-89.

Rosenberg, E. B., McCoy, J. L., Green, S. S., Donnelly, F. C., Siwarski, D. F., Levine, P. H., and Herberman, R. B. (1974) Destruction of human lymphoid tissue culture cell lines by human peripheral lymphocytes in 51-Cr release cellular cytotoxicity assays. J. Natl. Cancer Inst. 52, 345.

Rotanda, J., Garcia-Calvo, M., Bull, H. G., Geissler, W. M., McKeever, B. M., Willoughby, C. A., Thornberry, N. A., and Becker, J. W. (2001) The three-dimensional structure of human granzyme B compared to caspase-3, key mediators of cell death with cleavage specificity for aspartic acid in P1. Chemistry and Biology 8, 357-368.

Russell, J. H., and Ley, T. J. (2002) Lymphocyte-mediated cytotoxicity. Annu. Rev. Immunol. 20, 323-370.

Schwarer, A. P., Jiang, Y. Z., Deacock, S., Brookes, P. A., Barrett, A. J., Goldman, J. M., Batchlor, J. R., and Lechler, R. I. (1994) Comparison of helper and cytotoxic antirecipient T cell frequencies in unrelated bone marrow transplantation. Transplantation 58, 1198.

Shi, L., Kraut, R. P., Aebersold, R., and Greenberg, A. H. (1992) A natural killer cell granule protein that induces DNA fragmentation and apoptosis. J. Exp. Med. 175, 553-566.

Slezak, S. E., and Horan, P. K. (1989) Cell-mediated cytotoxicity: A highly sensitive and informative flow cytometric assay. J. Immunol. Methods 117, 205-214.

Smolewski, P., Bedner, E., Du, L., Hsieh, T. Wu, J. M., Phelps, D. J., and Darzynkiewicz, Z. (2001) Detection of caspases activation by fluorochrome-labeled inhibitors: Multiparameter analysis by laser scanning cytometry. Cytometry 44, 73-82.

Smolewski, P., Grabarek, J., Lee, B. W., Johnson, G. L., and Darzynkiewicz, Z. (2002) Kinetics of HL-60 cell entry to apoptosis during treatment with TNF-α or camptothecin assayed by the stathmo-apoptosis method. Cytometry 47, 143-149.

Smyth, M. J., Kelly, J. M., Sutton, V. R., Davis, J. E., Browne, K. A., Sayers, T. J., and Trapani, J. A. (2001) Unlocking the secrets of cytotoxic granule proteins. J. Leukoc. Biol. 70, 18-29.

Szekeres, J., Pacsa, A. S., and Pejtsik, B. (1981) Measurement of lymphocyte cytotoxicity by assessing endogenous alkaline phosphatase activity of the target cells. J. Immunol. Methods 40, 151-154.

Thornberry et al., Biochemistry, 1994, 33:3934-3940.

Thornberry, N. A., Rano, T. A., Peterson, E. P., Rasper, D. M., Timkey, T., Garcia-Calvo, M., Houtzager, V. M., Nordstrom, P. A., Roy, S., Vaillancourt, J. P., Chapman, K. T., and Nicholson, D. W. (1997) A combinatorial approach defines specificities of members of the caspase family and granzyme B. J. Biol. Chem. 272, 17907-17911.

Trapani (2001) Granzymes: A family of lymphocyte granule serine proteases, Genome Biology, 2, 3014.1-3014.7.

Trapani, A. J., and Sutton, V. R. (2003) Granzyme B: pro-apoptotic, antiviral and antitumor functions. Curr. Opin. Immunol. 15, 533-543.

Ward, P. W., Bonaparte, M. I., and Barker, E. (2004) HLA-C and HLA-E reduce antibody-dependent natural killer cell-mediated cytotoxicity of HIV-infected primary T cell blasts. AIDS 18, 1769-1779.

Yang, S., and Haluska, F. G. Treatment of melanoma with 5-fluorouracil or dacarbazine in vitro sensitizes cells to antigen-specific CTL lysis through perforin/granzyme-and Fas-mediated pathways. J. Immunol. 172, 4599-4608.
U.S. Patent Application Pub. No. 2004/0048327
U.S. Patent Application Pub. No. 2005/0136492
U.S. Pat. No. 5,543,396
U.S. Pat. No. 5,681,821
U.S. Pat. No. 5,916,877

All publications and patent documents cited herein are incorporated by reference herein, as though individually incorporated by reference.

The invention has been described with reference to various specific and preferred embodiments and techniques. However, it should be understood that many variations and modifications may be made while remaining within the spirit, and scope, of the invention.

1. A method for determining the viability of a population of cells, comprising:
combining with a sample that comprises a first population of cells a first fluorescent probe that is a membrane stain, a second fluorescent probe that is a vital stain, and a third fluorescent probe that is a cell-permeable apoptosis-detection probe that binds to active caspase enzymes;
and detecting the cells that bind to one or more of the probes so as to determine the viability of the population of cells.

2. The method of claim 1, further comprising combining a second population of cells with the sample after the first population of cells has been combined with the membrane stain and the membrane stain binding reaction has been stopped.

3. The method of claim 2, wherein the second population of cells comprises immune cells.

4. The method of claim 3, wherein the immune cells comprise lymphocytes.

5. The method of claim 3, wherein the immune cells comprise natural killer cells.

6. The method of claim 1, wherein the first population of cells is exposed to an experimental agent before the second or third probes are combined with the sample.

7. The method of claim 6, wherein the experimental agent is an infectious agent, a pharmaceutical agent, or radiation.

8. The method of claim 1, wherein the method is performed in a single container.

9. The method of claim 8, wherein the container is a test tube, a flask, a 96 well plate, or a tissue culture plate.

10. The method of claim 1, wherein the detection is performed using fluorescence spectroscopy, fluorescence microscopy, confocal fluorescence microscopy, fluorescence image analysis, flow cytometry, laser scanning cytometry, or a plate multi-well fluorescence reader.

11. The method of claim 10, wherein the detection is performed using flow cytometry.

12. The method of claim 1, further comprising combining with the sample at least one additional probe that is a cell permeable granzyme B specific detection probe.

13. The method of claim 1, further comprising combining with the sample at least one additional probe that is a cell permeable granzyme A specific detection probe.

14. The method of claim 1, wherein the membrane stain is a thiol-reactive membrane stain or an amine-reactive membrane stain.

15. The method of claim 14, wherein the membrane stain is carboxyfluorescein diacetate succinimidyl ester (CFSE).

16. The method of claim 1, wherein the vital stain is a cell-impermeant DNA stain.

17. The method of claim 16, wherein the vital stain is 7-aminoactinomycin D (7-AAD).

18. The method of claim 1, wherein the cell-permeable apoptosis-detection probe is sulforhodaminyl-L-valylalanylaspartylfluoromethyl ketone (SR-VAD-FMK), or an ester or a salt thereof.

19. A method for determining the viability of a population of cells, comprising:
combining with a sample that comprises a first population of cells carboxyfluorescein diacetate succinimidyl ester (CFSE);
combining a second population of cells with the sample after the first population of cells has been combined with the CFSE and the CFSE binding reaction has been stopped;
combining 7-aminoactinomycin D (7-AAD), and sulforhodaminyl-L-valylalanylaspartylfluoromethyl ketone (SR-VAD-FMK), or an ester or a salt thereof with the sample;
and detecting via flow cytometry the cells that bind to the CFSE, 7-AAD, and/or SR-VAD-FMK so as to determine the viability of the first population of cells.

20. A kit, comprising a first fluorescent probe that is a membrane stain, a second fluorescent probe that is a vital stain, and a third fluorescent probe that is a cell-permeable apoptosis-detection probe that binds to active caspase enzymes.

21. The kit of claim 20, further comprising instructions for using the kit to determine the viability of a population of cells using flow cytometry.

22. The kit of claim 20, further comprising a cell permeable granzyme B specific detection probe.

23. The kit of claim 20, further comprising a cell permeable granzyme A specific detection probe.

24. The kit of claim 20, wherein the probes are packaged separately.

25. The kit of claim 20, wherein the membrane stain is a thiol-reactive membrane stain or an amine-reactive membrane stain.

26. The kit of claim 20, wherein the membrane stain is carboxyfluorescein diacetate succinimidyl ester (CFSE), the vital stain is 7-aminoactinomycin D (7-AAD), and the cell-permeable apoptosis-detection probe is sulforhodaminyl-L-valylalanylaspartylfluoromethyl ketone (SR-VAD-FMK), or an ester or a salt thereof.
